(19) United States
(12) Patent Application Publication
(10) Pub. No.: US 2014/0253705 A1
(43) Pub. Date: Sep. 11, 2014

(54) ADAPTIVE SPECTRAL-COMPOSITION CONTROL

(71) Applicant: BOSTON SCIENTIFIC SCIMED, INC., MAPLE GROVE, MN (US)

(72) Inventors: John KUMMAILIL, Sherborn, MA (US); Arnold BUCK, Lowell, MA (US); Zhihua HE, Reading, MA (US); Nathaniel McCAFFREY, Hampton Falls, NH (US)

(73) Assignee: BOSTON SCIENTIFIC SCIMED, INC., MAPLE GROVE, MN (US)

(21) Appl. No.: 14/199,215

(22) Filed: Mar. 6, 2014

Related U.S. Application Data
(60) Provisional application No. 61/774,155, filed on Mar. 7, 2013.

(51) Int. Cl. H04N 9/68

(52) U.S. Cl. CPC H04N 9/68
USPC 348/68

(57) ABSTRACT

A method for adjusting color channel signals based on spectral variations in the signal associated with a known source. In an embodiment of the present disclosure, the method may include receiving a first signal having an intensity value from an electromagnetic radiation source. In an embodiment of the present disclosure, the method may also include producing one or more second signals such that a response-ratio value calculated based on the one or more second signals satisfies a response-ratio condition associated with a spectral region of the first signal.

### CROSS-REFERENCE TO RELATED APPLICATIONS

This application claims the benefit of priority from U.S. Provisional Application No. 61/774,155, filed on Mar. 7, 2013, the entirety of which is incorporated by reference herein.

### FIELD

Organic material (e.g., blood clots, tissue, and biological concretions such as urinary, biliary, and pancreatic stones) or inorganic material (e.g., components of medical devices or other foreign matter) may sometimes obstruct or otherwise be present within the body's anatomical lumens, such as the biliary tree. For example, biological concretions can develop in certain parts of the body, such as the kidneys, pancreas, and gallbladder. Minimally invasive medical procedures generally involve causing limited trauma to the tissues of the patient, and can be used to dispose of certain problematic biological concretions or similarly unwanted obstructions.

### BACKGROUND

An image detector can be configured to receive and convert light reflected from an object into several color channel signals that can be used to produce images of the object on a display. The light reflected from the object can be emitted onto the object from a light source. In known systems, the image detector produces a set of color channel signals where each color channel signal is associated with a band of wavelengths. For example each band of wavelengths can be centered around each of the primary colors—red, green, and blue. The color channel signals can be balanced to produce an aggregated image having a specific color-composition such as a white-balanced color-composition. For example, a white-balanced color-composition can be achieved by mixing equal portions of the primary colors through adjustments of one or more gain values associated with each of the color channel signals.

The responsiveness of an image detector to either a single pixel or a pixel array can, however, vary among its color channels based on changes in light source type (e.g., tungsten light source, fluorescent light source) because the light source types can have different spectral characteristics. Specifically, the color composition of images produced by an image detector can vary with different light source types because the responsiveness of the image detector for each spectral channel can vary with the different light source types. Accordingly, devices that have image detectors, such as video cameras or still cameras, are often configured to automatically adjust color channel signals using different sets of gain values based on light source type to achieve a target color-composition (e.g., white balance). These devices are typically configured to detect a light source type using a sensor, and subsequently select a pre-programmed set of gain values based on the light source type. The light-source-type sensing/feedback mechanism is critical to produce images with a target color-composition because devices such as video cameras and still cameras are often used in a variety of environments that can have different types of light sources.

In an environment where the light source is a known light source, however, the techniques for automatic adjustment of color-composition based on light source type are inappropriate. Thus, a need exists for a method and apparatus for adjusting color channel signals based on spectral variations in the signal associated with a known light source.

### SUMMARY

In one embodiment, a method may include receiving a first signal having an intensity value from an electromagnetic radiation source; and producing one or more second signals such that a response-ratio value calculated based on the one or more second signals satisfies a response-ratio condition associated with a spectral region of the first signal. The method may also include producing an image based on the one or more second signals, the spectral-composition of the image varying based on a relative strength of the one or more second signals, an image detector being configured to receive the first signal and produce the one or more second signals, the image detector being associated with an endoscope, modifying the one or more second signals in response to intensity level changes of the first signal; modifying the one or more second signals in response to a gain-intensity relationship, the gain-intensity relationship being a function of the response-ratio condition, and/or the response-ratio condition including a target response ratio value and a range of response-ratio values.

Aspects of the disclosure relate to one or more of: producing an image based on the one or more second signals; wherein the spectral-composition of the image may vary based on a relative strength of the one or more second signals; wherein an image detector is configured to receive the first signal and produce the one or more second signals; wherein the image detector is associated with an endoscope; modifying the one or more second signals in response to intensity level changes of the first signal; modifying the one or more second signals in response to a gain-intensity relationship; wherein the gain-intensity relationship is a function of the response-ratio condition; wherein the response-ratio condition includes a target response ratio value and a range of response-ratio values.

In another embodiment, a method may include emitting electromagnetic radiation toward an object at a first intensity level and at a second intensity level; receiving a spectral region of the electromagnetic radiation reflected from the object at the first intensity and at the second intensity; determining a response-ratio value at the first intensity level and a response-ratio value at the second intensity level to define a response-intensity relationship; receiving a response-ratio condition; and defining a gain-intensity relationship based on the response-intensity relationship and the response-ratio condition. The method may also include the response-ratio condition being a target response-ratio condition, the response-ratio condition being a target range of response-ratio values, an electromagnetic source being configured to emit the electromagnetic radiation, and/or an image detector being configured to receive the spectral region of the electromagnetic radiation.

In a further embodiment, a method may include changing an intensity level of an electromagnetic radiation source emitting electromagnetic radiation onto an object; receiving an intensity defining signal associated with the intensity level of the electromagnetic radiation source; receiving an image detector signal associated with a spectral region of electromagnetic radiation being reflected from the object; and modifying the image detector signal based on a gain value associated with the image detector signal, the gain value being based on the indicator and the gain-intensity relationship. The method may also include calculating the gain value using the intensity defining signal prior to modifying the image detector signal, calculating the gain value includes using a distance value associated with a distance between the electromagnetic radiation source and the object, receiving the gain value prior to modifying the image detector signal, modifying the gain value prior to modifying the image detector signal, and/or changing of the intensity level is triggered by a change in the intensity defining signal.

### BRIEF DESCRIPTION OF THE DRAWINGS

FIG. 1 is a schematic diagram that illustrates an electromagnetic (EM) radiation source and an image detector in communication with a control unit, according to an exemplary embodiment of the present disclosure.

FIG. 2 is a schematic diagram that illustrates a variation of the device shown in FIG. 1, according to an exemplary embodiment of the present disclosure.

FIG. 3A is a schematic graph that illustrates a response-intensity relationship that can be used to produce a gain-intensity relationship, according to an exemplary embodiment of the present disclosure.

FIG. 3B is a schematic graph that illustrates a gain-intensity relationship produced based on the response-intensity relationship shown in FIG. 3A, according to an exemplary embodiment of the present disclosure.

FIG. 3C is a schematic graph that illustrates response-ratio values calculated based on image-detector signals that have been modified based on the gain-intensity relationship shown in FIG. 3B, according to an exemplary embodiment of the present disclosure.

FIG. 4 is a flowchart that illustrates a method for creating a gain-intensity relationship based on a response-intensity relationship and a response-ratio condition, according to an exemplary embodiment of the present disclosure.

FIG. 5 is a flowchart that illustrates a method for using a gain-intensity relationship, according to an exemplary embodiment of the present disclosure.

FIG. 6 is a schematic diagram that illustrates a gain-intensity table that can be used to modify an image detector signal, according to an exemplary embodiment of the present disclosure.

### DETAILED DESCRIPTION

An image detector can be configured to receive and convert electromagnetic (EM) radiation reflected from an object into one or more image-detector signals that can be used to produce one or more images of the object on a display. In some embodiments, the EM radiation reflected from the object can be converted into an image-detector signal with the aid of a processing element. The EM radiation can be reflected (e.g., scattered) from the object after being emitted towards the object from one or more EM radiation sources. EM radiation can include, for example, radio waves, microwaves, terahertz radiation, infrared radiation, visible light, ultraviolet radiation, x-rays, gamma rays, and/or so forth.

One or more of the image-detector signals produced by the image detector can be associated with a spectral region of EM radiation such as a specific spectral band (e.g., a band of wavelengths centered around red, or green, etc.). EM radiation emitted from an EM radiation source can be referred to as source EM radiation and the EM radiation reflected from an object can be referred to as reflected EM radiation. Reflected EM radiation can include EM radiation that is scattered (e.g., diffusely scattered) from, for example, a body lumen or object within a body lumen.

An image detector can have a responsiveness (also can be referred to as sensitivity) that varies with changes in intensity level of EM radiation received at the image detector (e.g., reflected EM radiation) and with changes in EM radiation source type (e.g., spectral characteristics). Moreover, the responsiveness of the image detector to different spectral regions of EM radiation (e.g., wavelength bands) can be different as a function of the intensity level of EM radiation received at the image detector. Consequently, the spectral-composition (e.g., color-composition) of images based on image-detector signals associated with different spectral regions of EM radiation can vary with changes in the intensity level of reflected EM radiation. In environments where the EM radiation source type and characteristics (e.g., spectral characteristics or color temperature) are known or specified, variations in spectral-composition of the resulting images can predominantly be caused by intensity level changes.

The responsiveness of the image detector can be characterized in terms of response-ratio values and can be associated with the intensity level of EM radiation received at the image detector in a response-intensity relationship. In some embodiments, a response-intensity relationship illustrates the relationship between response-ratio values as a function of intensity values. A response-ratio value can be calculated as a ratio of a value associated with an image-detector signal associated with a first spectral region of EM radiation to a value associated with an image-detector signal associated with a second spectral region of EM radiation. For example, the response-ratio value can be calculated based on voltage level values, current level values, and/or digital numbers (DN) associated with one or more image detector signals. In some embodiments, the response-ratio can be referred to as an intensity ratio.

A spectral region of EM radiation, in some embodiments, can be a continuous or a discontinuous (e.g., periodic, irregular) band of wavelengths centered around a specific wavelength. For example, the band of wavelengths can be centered around a visible color wavelength such as a red wavelength or a green wavelength. A band of wavelengths centered around a wavelength can be referred to as a channel for convenience. A band of wavelengths centered around a visible color wavelength can be referred to as a color channel for convenience.

A response-intensity relationship can be used to define a gain-intensity relationship that can be used to modify (e.g., attenuate, amplify) or leave unchanged, one or more image-detector signals such that an image produced based on the modified image-detector signals can have a specified spectral-composition. The gain-intensity relationship can also be defined based on one or more conditions such as, for example, a response-ratio condition. The response-ratio condition can be defined based on a target spectral-composition (e.g., white-balanced color-composition) or a response-ratio profile that includes several response-ratio target values. For example, a gain value associated with an image-detector signal can be modified based on a gain-intensity relationship such that an image based on the modified image-detector signal can have a specified spectral-composition (e.g., white-balanced color-composition) at a specified reflected EM radiation intensity level. In some embodiments, the image-detector signal can be modified using a control module that has access to the gain-intensity relationship.

The gain-intensity relationship (and the response-intensity relationship) used for modifying the image-detector signal can be based on the intensity level associated with EM radiation from the EM radiation source. This is because the intensity level of reflected EM radiation can be correlated to (e.g., proportionally related to, or non-linearly related to) the intensity level of source EM radiation. Accordingly, the image-detector signal can be modified based on an intensity-defining signal used to define (e.g., directly or indirectly triggering) the intensity level of the EM radiation source rather than, for example, based on a feedback signal from a light intensity sensor configured to detect the intensity level of EM radiation from the EM radiation source. In some embodiments, the EM radiation source can be substantially free from interference from unneeded and/or unwanted EM radiation sources (e.g., ambient light). For example, the methods and apparatus described herein can be used in a medical device for investigation within a cavity of a body (e.g., endoscope) where the EM radiation source is known and substantially the only source of EM radiation. This can be referred to for convenience as a dark environment.

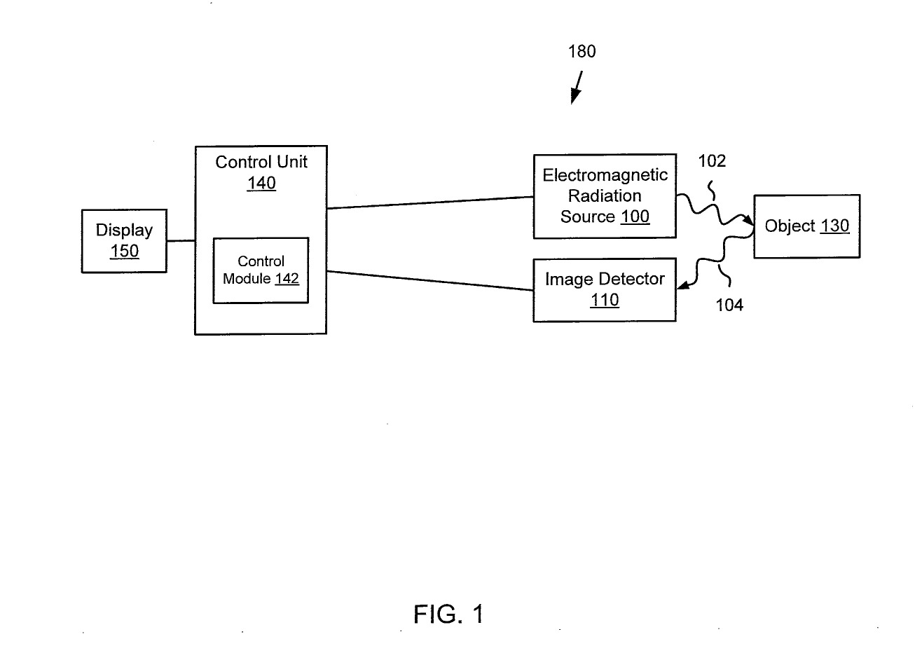

FIG. 1 is a schematic diagram that illustrates a device 180 that includes an electromagnetic (EM) radiation source 100 and an image detector 110 in communication with a control unit 140, according to an embodiment of the invention. The EM radiation source 100 can be configured to illuminate an object 130 by emitting EM radiation 102 (referred to as source EM radiation) onto the object 130. The EM radiation source 100 can be any type of EM radiation source 100 configured to emit EM radiation. For example, the EM radiation source 100 can be a light emitting diode (LED) that emits a specific spectrum of EM radiation, a tungsten-based light, an infrared generator, and so forth.

The EM radiation source 100 can be configured to emit source EM radiation 102 at a specified intensity level in response to an intensity-defining signal. The intensity-defining signal can be any type signal and/or an indicator used to define and/or trigger the intensity level of source EM radiation 102. For example, the intensity-defining signal can be a current.

The image detector 110 can be configured to receive at least a portion of the source EM radiation 102 that is reflected 104 (referred to as reflected EM radiation 104) from the object 130. The image detector 110 can be further configured to produce one or more image-detector signals based on the reflected EM radiation 104. The image-detector signals can then be used to produce an image of the object 130 on the display 150. One or more of the image-detector signals produced by the image detector 110 can be associated with a defined spectral region of EM radiation such as a specific color channel (e.g., red, green). The spectral-composition of the images produced can vary based on, for example, the relative strength or values of the image detectors signals and/or the spectral region(s) of EM radiation associated with each the image-detector signals. The image-detector signals can be, for example, analog signals (e.g., voltage-based signal, frequency-based signal) or digital signals.

The image detector 110 can be any type of image detector that receives and converts EM radiation into an image-detector signal to display an image. Although not shown, the image detector 110 can include, for example, a lens or set of lenses, and/or can include one or more charge coupled devices (CCD), complimentary metal-oxide-semiconductor (CMOS) sensors, active pixel sensors, thermal imaging sensors, video camera tubes, gamma camera sensors, x-ray sensors, etc. The image detector 110 can also include one or more techniques for separating and/or detecting different spectral regions of EM radiation that can be associated with a color channel, such as a Bayer sensor, a Foveon X3 sensor, and/or dichroic prism. Different spectral regions of EM radiation can be associated with one or more image-detector signals that can each be produced by, for example, different CCDs within image detector 110 or multiple image detectors (not shown).

A control module 142 within the control unit 140 can modify the signal(s) from the image detector 110 in response to intensity level changes of source EM radiation 102 emitted from the EM radiation source 100 such that images displayed based on the image-detector signal(s) can have, for example, a specified spectral-composition (e.g., white-balanced color-composition). The control module 142 can be associated with (e.g., stored on) a memory (not shown) and/or processed using a processor (not shown). In some embodiments, the control module can be a hardware and/or software control module 142.

In some embodiments, the control module 142 can modify one or more image-detector signals based on a gain-intensity relationship. The gain-intensity relationship can be defined such that an image produced based on an image-detector signal(s) modified based on the gain-intensity relationship can have a specified spectral-composition. The gain-intensity relationship can be defined based on a response-intensity relationship and one or more conditions such as, for example, a response-ratio condition. The response-ratio condition can be defined based on the specified spectral-composition (also can be referred to as a target spectral-composition) or a response-ratio profile that includes several response-ratio target values. For example, a gain value associated with an image-detector signal can be modified based on a gain-intensity relationship to produce a modified image-detector signal. An image produced at the display 150 based on the modified image-detector signal can have a specified spectral-composition (e.g., white balance color-composition) at a specified reflected EM radiation intensity level.

The response-intensity relationship can be a characterization of the responsiveness of the image detector in terms of response-ratio values and can be associated with the intensity level of EM radiation received at the image detector 110. In some embodiments, a response-intensity relationship indicates the relationship between response-ratio values as a function of intensity values. A response-ratio value can be calculated as a ratio of a signal associated with a first spectral region of EM radiation (e.g., red color channel—a band of wavelengths centered around a red wavelength) to a signal associated with a second spectral region of EM radiation (e.g., blue color channel—a band of wavelengths centered around a red wavelength).

A response-intensity relationship and/or gain-intensity relationship defined based on the response of the image detector 110 and/or the output of the EM radiation source 100 can be used by one or more separate devices (not shown) configured with an image detector (not shown) substantially similar to image detector 110 and/or an EM radiation source (not shown) substantially similar to EM radiation source 100.

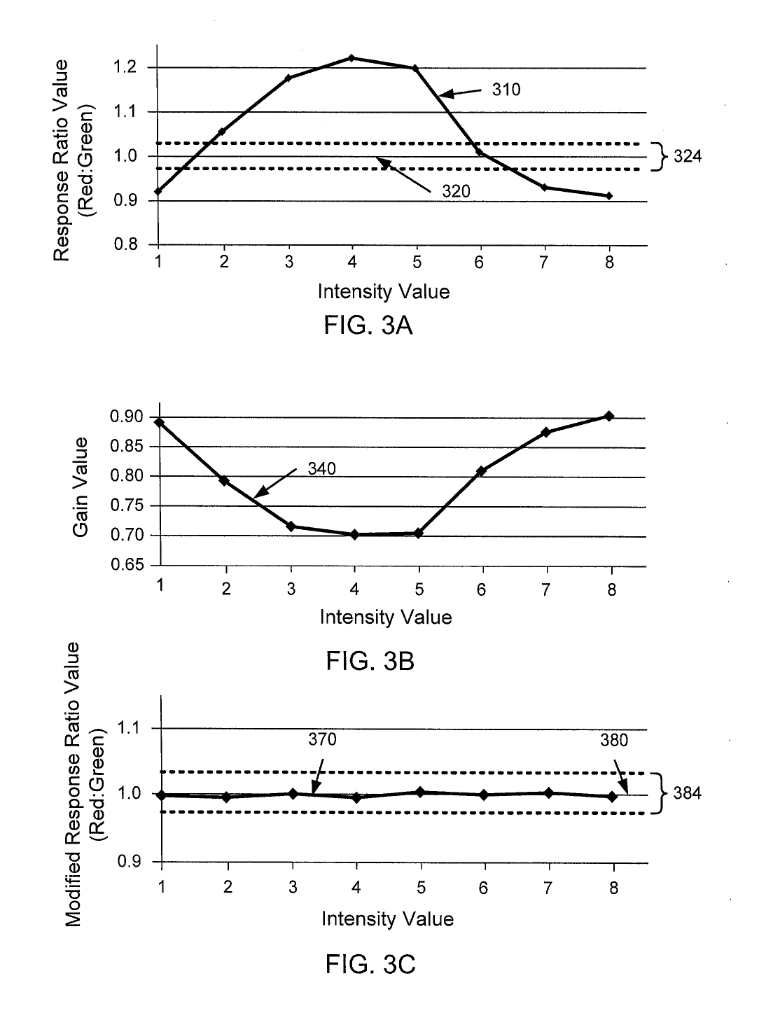

In some embodiments, a gain-intensity relationship and/or a response-ratio relationship can be defined for any combination of image-detector signals produced by image detector 110. For example, a first gain-intensity relationship can be defined based on a response-ratio relationship associated with a first and a second image-detector signal, and a second gain-intensity relationship can be defined based on a response-ratio relationship associated with the second and a third image-detector signal. A detailed example of a gain-intensity relationship defined by a response-intensity relationship associated with an image detector and an EM radiation source is described in connection with FIG. 3.

In some embodiments, the EM radiation source 100, such as that shown in FIG. 1, can substantially be the only source of EM radiation that is incident on the object 130 and/or received by the image detector 110. For example, the EM radiation source 100 and the image detector 110 can be coupled to a component (not shown) of a medical device (e.g., endoscope) where the EM radiation source 100 is substantially the only source of EM radiation within the illuminated environment (e.g., a body lumen). Hence, the reflected EM radiation 104 (or any EM radiation received at the image detector 110) can be substantially free from interference from other EM radiation sources (not shown), such as ambient light, within the illuminated environment (e.g., a body lumen). These other unneeded and/or unwanted EM radiation sources can be referred to as renegade EM radiation sources or unknown EM radiation sources. In some embodiments, EM radiation sources are considered unknown/renegade radiation sources because the control module 142 does not have access to (e.g., cannot receive) the intensity-defining signal(s) associated with the unknown/renegade EM radiation source(s).

The gain-intensity relationship used for modifying one or more image-detector signals associated with the image detector 110 can be based on a characterization of a responsiveness of the image detector 110 to changes in intensity level of source EM radiation 102 emitted from the EM radiation source 100. The image-detector signal can be modified based on an intensity-defining signal used to define the intensity level of the EM radiation source 100 rather than based on a feedback signal from, for example, a light intensity sensor (not shown) configured to detect the intensity level of source EM radiation 102 emitted the EM radiation source 100. This type of gain-intensity relationship can be produced because the intensity level of reflected EM radiation 104 can be correlated to (e.g., proportionally related to, or non-linearly related to) the intensity level of source EM radiation 102 and because the EM radiation source 100 can be the only source EM radiation within the illuminated environment.

In some embodiments, an additional EM radiation source (not shown) in addition to the EM radiation source 100 can be configured to emit EM radiation (e.g., emit EM radiation towards object 130) so long as the control module 142 has access to (e.g., can receive) the intensity-defining signal(s) associated with the additional EM radiation source(s). This allows image-detector signals to be modified by the control module 142 based on the intensity-defining signal(s). In other words, the control module 142 can modify an image-detector signal based on intensity-defining signals associated with one or more EM radiation sources without using, for example, a feedback signal from a light intensity sensor (not shown) configured to detect the intensity level of source EM radiation 102 and/or reflected EM radiation 104. In some embodiments, the control module 142 can be configured to have access to all intensity-defining signals from EM radiation sources within a dark environment.

Although not shown in FIG. 1, the image detector can also include one or more processors and/or components (e.g., circuit components) that can receive, produce and/or process image-detector signals such that one or more image-detector signals that can be produced and/or sent to the control unit 140 for further processing. The image detector 110 can be configured to produce one or more image-detector signals that can be associated with one or more spectral regions of EM radiation (e.g., a signal associated with a band of wavelengths centered around visible green wavelength). In some embodiments, the image-detector signals can filter EM radiation received at the image detector to produce an image-detector signal associated with a specified spectral region of the EM radiation.

In some embodiments, the EM radiation source 100 can be configured to emit EM radiation at a specified intensity level in response to an intensity-defining signal such as a current. In some embodiments, the current can be triggered by an intensity-defining signal having an indicator sent from, for example, the control unit 140 or control module 142. In some embodiments, the intensity-defining signal can be triggered and/or produced based on a user-triggered action such as a turning of a knob (not shown) or an actuation of a button (not shown). In some embodiments, the knob or button can trigger a specific intensity level (e.g., low, medium, high). Alternatively, the intensity-defining signal can be an electronic signal produced and/or modified using the control module 142.

In some embodiments, the EM radiation source 100 can be configured to emit EM radiation associated with a specified spectral region and/or at a specified intensity level. For example, the EM radiation source 100 can be an EM radiation source that only emits EM radiation associated with a specific spectral region of EM radiation. In some embodiments, for example, the intensity-defining signal can be used to select one of several EM radiation sources (not shown) from a group of radiation sources and can cause the selected EM radiation source to emit EM radiation at a specified intensity level.

The EM radiation source 100 and/or image detector 110 can be coupled to different portions of the component in some embodiments. For example, if coupled to the component within the endoscope, the EM radiation source 100 and/or image detector 110 can be coupled to a distal portion or a proximal portion of the component. The EM radiation source 100, for example, can be configured to emit EM radiation from the proximal portion of the component through a fiber optic onto an object and the image detector 110 can be coupled to the distal portion of the component to receive EM radiation reflected from the object.

In some embodiments, the control unit 140 and/or display 150 can be coupled to a housing. In some embodiments, the EM radiation source 100 and/or image detector 110 can be removably coupled to the housing. In some embodiments, the EM radiation source 100 and/or image detector 110 can be in communication with the control unit 140 wirelessly. In other embodiments, the EM radiation source 100 and/or image detector 110 can be in communication with the control unit 140 via a fiber or bundle of fibers.

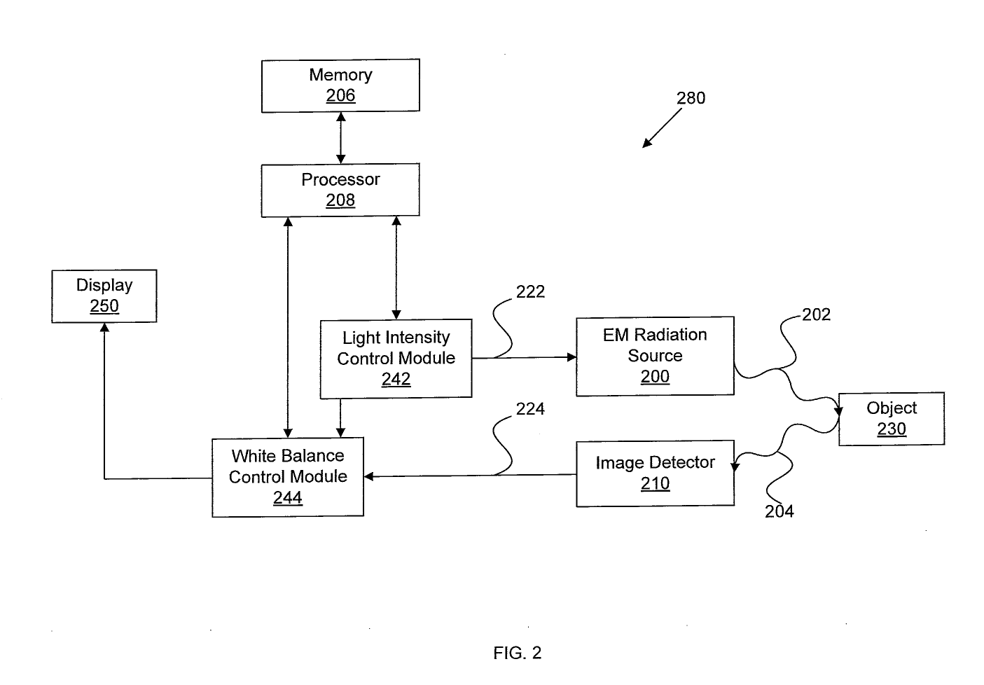

In some embodiments, the control module 142 and/or a separate control module (not shown) associated with the control unit 140 can be configured to define an intensity-defining signal. In some embodiments, one or more of the features and/or functions of the control unit 140 and/or the control module 142 can be integrated into the image detector 110, the EM radiation source 100, and/or the component. Likewise, one or more of the features and/or functions of the image detector 110, the EM radiation source 100, and/or the component can be integrated into the control unit 140 and/or the control module 142. In some embodiments, signals can be sent (not shown) from the EM radiation source 100 to the image detector 110, and vice versa. FIG. 2 is a schematic diagram that illustrates one such variation of the device 180 shown in FIG. 1, according to an embodiment of the invention.

FIG. 2 is a schematic diagram that illustrates a device 280 including an EM radiation source 200 in communication with a light intensity control module 242, according to an embodiment of the invention. In this embodiment, an intensity-defining signal 222 is received at the EM radiation source 200 from the light intensity control module 242. The device 280 includes a processor 208 configured to calculate one or more gain values based on the intensity-defining signal 222 and based on a gain-intensity relationship stored in a memory 206. The gain value(s) are sent to the image detector 210 where one or more image-detector signals can be modified (e.g., using a processor (not shown)). The image detector 210 can send the modified image-detector signal(s) 224 to the white balance control module 244 for further processing.

In some embodiments, the light intensity control module 242 can be configured to calculate and/or send the gain value(s) to the image detector 210 before, after, or at the same time that the intensity level of the EM radiation source 200 is adjusted. For example, the light intensity control module can be configured to calculate and/or send the gain value(s) to the image detector 210 after, for example, a specified period of time has elapsed to account for delay in changing the intensity level of the EM radiation source 200 in response to the intensity defining signal 222.

In a variation of FIG. 2, rather than sending modified image-detector signal(s) 224 to the white balance control module 244, the image detector 210 can send unmodified image-detector signal(s) to the EM radiation source 200 where the unmodified image-detector signals can be modified at the processor 208 based on the gain value(s). The EM radiation source 200 can then send the modified image-detector signal(s) (not shown) to the control unit 240 for further processing.

FIGS. 3A, 3B, and 3C are schematic graphs of various parameter values (shown as y-axis values in the graphs) associated with an image detector as a function of an intensity value (shown as x-axis values in the graphs) of an EM radiation source, according to an embodiment of the invention. FIG. 3A illustrates a response-intensity relationship 310 that can be used to produce a gain-intensity relationship 340, shown in FIG. 3B. The gain-intensity relationship can be used to modify image-detector signals produced by the image detector. The gain-intensity relationship 340 can be produced as a function of a response-ratio condition. In this embodiment, the response-ratio condition includes two components—a target response-ratio value of 1.0 and an acceptable range of response-ratio values. In this embodiment, the target response-ratio value is a white-balanced target response-ratio value. FIGS. 3A and 3C illustrate the target response-ratio value, 320 and 380, respectively, and the acceptable range of response-ratio values, 324 and 384, respectively, defined as part of the response-ratio condition. FIG. 3C illustrates response-ratio values calculated based on image-detector signals that have been modified based on the gain-intensity relationship 340, shown in FIG. 3B, to produce a modified response-intensity relationship 370 that satisfies the response-ratio condition.

Although the intensity values associated with the EM radiation source in these graphs are arbitrary values used for illustrative purposes, in some embodiments, the intensity values can be, for example, values associated with an intensity-defining signal such as a value of a voltage or a value of a current supplied to the EM radiation source. In some embodiments, the intensity values can be, for example, the intensity level of the EM radiation source as measured in lumens (Im) or candela (cd) per square foot (ft²). In some embodiments, the intensity values can be an intensity level setting associated the EM radiation source. More details related to FIGS. 3A, 3B, and 3C are described below.

As shown in FIG. 3A, the response-intensity relationship 310 illustrates a non-linear relationship between response-ratio values (calculated based on image-detector signals associated with the image detector) as a function of intensity values associated with the EM radiation source. The response-ratio values in the response-intensity relationship 310 are calculated based on an intensity level of a red color channel (e.g., a band of wavelengths centered around a red wavelength) and an intensity level of a green color channel (e.g., a band of wavelengths centered around a green wavelength) associated with the image detector at intensity values from 1 to 8 (x-axis) to produce ratios for values for the red color channel to values for the green color channels. In some embodiments, the response-ratio values can be calculated based on voltage level values, current level values, and/or DNs associated with each of the color channels.

Although FIGS. 3A, 3B, and 3C are related to a green and red color channels, these particular bands of wavelengths are described for convenience. The principles described and/or illustrated in connection with these figures can be similarly applied to various bands of continuous or a discontinuous (e.g., periodic, irregular) bands of wavelengths that can be centered around one or more specific wavelengths (e.g., a wavelength in the IR spectral region, a wavelength in the UV spectral region, etc.).

As shown in FIG. 3A, only a few response-ratio values on the response-intensity relationship 310 curve will meet the response-ratio condition. At an intensity value of 4, for example, the response-ratio value is more than 1.2. This can be an indicator that the sensitivity of the image detector to EM radiation associated with the red color channel at the intensity value of 4 is higher than that of the image detector to EM radiation associated with the green channel at the intensity value of 4. Thus, an image produced based on this mix of image-detector signals will be unacceptably unequal (not within a range around the white-balanced point) with respect to these two primary colors. The response-ratio value of 1.05 at the intensity value of 6 is within the acceptable range of response-ratio values 324 and close to the target response-ratio value of 1.0 (line 320), which, in this embodiment, is a white-balanced response-ratio target value.

The response-intensity relationship 310 shown in FIG. 3A can be produced based on images captured (also can be referred to as frames) using the image detector at each of the intensity values of the EM radiation source. The intensity level of the red color channel and the intensity level of the green color channel associated with the captured images can then be used to calculate the response-ratio values associated with the intensity values. Specifically, the response-ratio values can be calculated based on, for example, image-detector signals used to produce the captured images or based on an analysis of colors (e.g., color channels) associated with the captured images after being produced. In some embodiments, response-ratio values can be calculated based on different types of signals (e.g., current values) and different spectral regions of EM radiation.

FIG. 3B is a schematic graph that illustrates a gain-intensity relationship 340 produced based on the response-intensity relationship 310, shown in FIG. 3A, and a response-ratio condition. The gain values in the gain-intensity relationship 340 shown in FIG. 3B are associated with the intensity level of the red color channel of the response-ratio values. Each gain value shown in FIG. 3B is defined such that when the gain value is used to modify an image-detector signal associated with the red color channel the resulting modified response-ratio value will satisfy the response-ratio condition (response-ratio conditions are shown in FIGS. 3A and 3C).

Although in this embodiment, the gain-intensity relationship was defined based on gain values associated with the red color channel, in some embodiments, a gain-intensity relationship can be defined based on gain values associated with the green color channel or different color channel (e.g., blue color channel). In some embodiments, a combination of a gain-intensity relationships for both the green and red color channels can be defined and used to modify image-detector signals associated with the green and red color channels.

FIG. 3C illustrates response-ratio values calculated based on image-detector signals that have been modified based on the gain-intensity relationship 340 (shown in FIG. 3B) to produce a modified response-intensity relationship 370 that satisfies the response-ratio condition. For simplicity, FIG. 3C only includes response-ratio values for each of the intensity values included in the gain-intensity relationship 340 shown in FIG. 3B. For other embodiments, any number of response-ratio values are possible.

In some embodiments, interpolated gain values can be calculated based on the gain-intensity relationship 340. The interpolated gain values can used to modify one or more image-detector signals from an image detector to produce a response-ratio value that satisfies the response-ratio condition shown in FIGS. 3A and 3C. For example, one or more image-detector signals produced at an intensity value of 2.5 can be modified based on an interpolated gain value calculated based on the gain-intensity relationship. The interpolation techniques that can be used include, for example, a linear interpolation technique, a polynomial interpolation technique, a spline interpolation technique, a Bezier interpolation technique, and so forth. In some embodiments, gain values can be calculated based on an extrapolation of the gain-intensity relationship.

The image detector and/or EM radiation source used to produce the response-intensity relationship 310 (FIG. 3A) and the gain-intensity relationship 340 (FIG. 3B) can be different than the image detector and/or EM radiation source used to produce the modified response-intensity relationship 370 shown in FIG. 3C. For example, in some embodiments, the gain-intensity relationship can be defined using an archetypical image detector/EM radiation source and then implemented in multiple image detectors/EM radiations sources based on the archetypical image detector/EM radiation source.

In some embodiments, a gain-intensity relationship can be defined specifically for an image detector/EM radiation pair. Because binning EM radiation sources and/or image detectors based on characteristics related to, for example, manufacturing variations can require some effort, specifically defining a gain-intensity relationship for an image detector/EM radiation pair regardless of their characteristics can be cost effective for some embodiments. For example, EM radiation sources (e.g., LEDs) of the same type can have varying chromaticity and/or intensity and image detectors (e.g., CCDs) of the same type can have different responsiveness to EM radiation.

Although FIGS. 3A, 3B, and 3C are related to response-ratio values and gain values associated with green and red color channels, in some embodiments, response-intensity relationships, gain-intensity relationships, response-ratio values, and/or gain values can be produced for different combinations of spectral regions of EM radiation (e.g., non-visible spectral regions of EM radiation). For example, a first gain-intensity relationship can be associated with a response-intensity relationship based on a first color channel and a second color channel, and a second gain-intensity relationship can be associated with a response-intensity relationship based on the second color channel and a third color channel. The first gain-intensity relationship and second gain-intensity relationship can be based on the same or different intensity values. The color channels can be associated with colors other than primary colors or other bands within different spectral regions (e.g., IR, UV, etc.).

In some embodiments, response-intensity relationships, gain-intensity relationships, or any calculations associated with these relationships can be based on more than one EM radiation source. For example, a gain-intensity relationship can be defined based on a ratio of intensity levels of two EM radiation sources. If the ratio of the intensity levels can be changed, multiple gain-intensity relationships can be configured for each ratio of intensity levels associated with EM radiation sources. In some embodiments, a gain-intensity relationship can be defined based on, for example, two EM radiation sources where an intensity level of one of the EM radiations sources is constant and an intensity level of the other EM radiation source can vary.

In some embodiments, a set of response-intensity relationships and gain-intensity relationships can be produced where each response-intensity relationship and gain-intensity relationship is associated with a particular set of environmental conditions. The environmental conditions can include, for example, a temperature condition and/or a pressure condition. The set of gain-intensity relationships and associated set of environmental conditions can be uploaded to or stored at a control module associated with an image detector and EM radiation source. The control module can be configured to select a gain-intensity relationship from the set of gain-intensity relationships based on environmental conditions. The environmental conditions can be detected using one or more sensors (e.g., temperature sensor, pressure sensor) in communication with the control module. In some embodiments, a set of gain-intensity relationship can be defined based on changes in distance of the image detector and/or EM radiation source from an object.

Although in this embodiment, the response-ratio condition was defined based on a target response-ratio value of 1.0 to produce a balanced relationship between the intensity levels of the green and red channels produced by the image detector, in some embodiments, the response-ratio condition can be defined based on a response-ratio profile that can be linear or non-linear. Gain values can be calculated, for example, such that image-detector signals modified based on the gain values can result in a modified response-intensity relationship that has increasing response-ratio values over a first range of intensity values and decreasing response-ratio values over a second range of intensity values. In some embodiments, a first response-ratio condition can be associated with a first range of intensity values and a second response-ratio condition can be associated with a second range of intensity values.

In some embodiments, different parameters and/or algorithms other than a gain value can be used to modify image-detector signals produced by an image detector. The gain-intensity relationship can be defined accordingly. In some embodiments, for example, if an image detector is a digital signal, the image-detector signal can be modified (e.g., attenuated, amplified) using an algorithm that includes, for example, processor-executable instructions. The algorithm can modify the digital signal based on changes in intensity value associated with an EM radiation source. In some embodiments, if an image-detector signal is a frequency-based signal, the gain-intensity relationship can be defined to modify the image-detector signal by modulating the frequency of the image-detector signal with changes in intensity value.

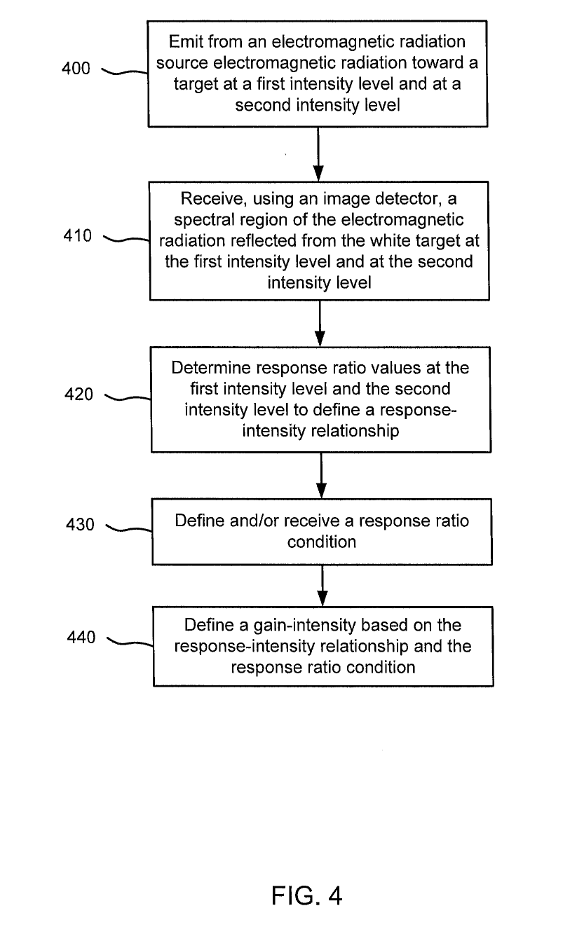

FIG. 4 is a flowchart that illustrates a method for creating a gain-intensity relationship based on a response-intensity relationship and a response-ratio condition, according to an embodiment of the invention. The flowchart illustrates that electromagnetic radiation is emitted from an electromagnetic radiation source toward a target (e.g., white target, object) at a first intensity level and at a second intensity level at 400. The EM radiation source can be any type of EM radiation source configured to emit EM radiation.

A spectral region of the electromagnetic radiation reflected from the target at the first intensity level and the second intensity level is received at an image detector at 410. The image detector can be any type of image detector that can be used to receive and convert EM radiation into an image-detector signal that can be used to display an image.

Response-ratio values at the first intensity level and the second intensity level of the EM radiation source are determined to define a response-intensity relationship at 420. The response-ratio values can be calculated as a ratio of a signal associated with a first spectral region of reflected EM radiation to a signal associated with a second spectral region of reflected EM radiation. For example, the response-ratio values can be calculated based on indicators of the intensity levels of two separate or overlapping spectral regions of reflected EM radiation.

A response-ratio condition is then received or defined at 430. The response-ratio condition can be a target response-ratio value or a target range of response-ratio values that define a spectral-composition for images to be produced based on signals associated with the image detector. In some embodiment, the response-ratio condition can be a response-ratio target profile or a white balance target value.

A gain-intensity relationship is then defined based on the response-intensity relationship and based on one or more response-ratio conditions at 440. Gain values in the gain-intensity relationship can be defined such that response-ratio values calculated based on image-detector signals modified based on the gain values will satisfy the response-ratio condition(s). For example, a gain value can be defined such that an image-detector signal used in a response-ratio value calculation can be modified such that the response-ratio value will satisfy a response-ratio condition.

When producing the gain-intensity relationship, the EM radiation source and/or the image detector can be positioned to mimic their relative positions when the EM radiation source and the image detector or substantially similar EM radiation sources and/or image detectors are implemented in an application. For example, if used in an endoscope application, the EM radiation source and the image detector can be very close to one another and the angle of incidence of EM radiation emitted from the source and received at the image detector can be relatively small (e.g., less than 20 degrees). When producing the gain-intensity relationship, the EM radiation source and/or the image detector can be placed to produce a substantially similar angle of incidence to that associated with the endoscope application. Also, in some embodiments, the image detector and/or the EM radiation source can be positioned at a distance from the white target that is a typical distance from an object in an actual application.

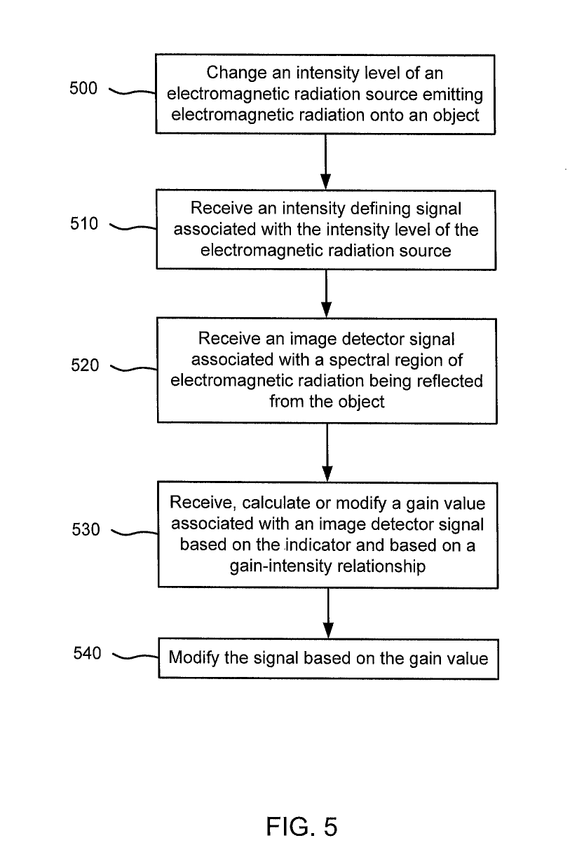

FIG. 5 is a flowchart that illustrates a method for using a gain-intensity relationship, according to an embodiment of the invention. The gain-intensity relationship can be, for example, a gain-intensity relationship defined using the method illustrated in FIG. 5. The gain-intensity relationship can be used in an apparatus such as, for example, that shown in FIG. 1 or FIG. 2.

As shown in FIG. 5, an intensity level of an electromagnetic radiation source emitting EM radiation onto an object is changed. The change can be triggered by, for example, an entity (e.g., person, computer) using the EM radiation source. In some embodiments, the change in the intensity level can be detected by, for example, a control module associated with the EM radiation source. Alternatively, the change in intensity level can be triggered by a change in an intensity-defining signal such as a change in a current level.

An intensity-defining signal associated with the intensity level of the electromagnetic radiation source is received at 510. The intensity-defining signal can be received at, for example, a control module associated with the EM radiation source. The intensity-defining signal can be a first intensity-defining signal associated with a second intensity-defining signal that is triggered by the first intensity-defining signal. For example, the second intensity-defining signal can be a current value associated with a current to an EM radiation source and the first intensity-defining signal can be an intensity level defined in lumens that triggers the current to the EM radiation source.

An image-detector signal associated with a spectral region of EM radiation being reflected from the object is received at 520. The image-detector signal can be one of several image-detector signals produced by an image detector. In some embodiments, the image-detector signal can be received at a control module associated with the image detector and the EM radiation source.

A gain value associated with the image-detector signal is then received, calculated and/or modified based on the intensity-defining signal at 530. In some embodiments, for example, the gain value can be received/retrieved based on the intensity-defining signal by a control module from a database (e.g., table in a database) stored in a memory associated with the control module. The database can be produced based on a gain-intensity relationship. The gain-intensity relationship can be defined based on a response-intensity relationship and a response-ratio condition.

In some embodiments, the gain value can be calculated using the intensity-defining signal and based on an equation or algorithm (e.g., set of processor-readable instructions) defining a gain-intensity relationship. The gain value can be an interpolated or extrapolated gain value. In some embodiments, a gain value associated with the image-detector signal can be modified (e.g., replaced) based on a received/retrieved and/or calculated gain value.

In some embodiments, the receiving, calculating, and/or modifying can be triggered in response to the receiving of the intensity-defining signal at 510 or the receiving of the image-detector signal at 520. A control module, in some embodiments, can be configured to wait until a triggering event, such as a change in intensity level of the EM radiation source, before, for example, querying a database for a gain value. In some embodiments, the gain-intensity relationship used to define the gain value can be a function of environmental conditions.

The image-detector signal is modified based on the gain value at 540. In some embodiments, the image-detector signal can be modified by a control module associated with the image detector and/or the EM radiation source. In some embodiments, the image-detector signal modified based on the gain value can be used to produce an image on a display.

In some embodiments, the image-detector signal can be further modified based on a signal (e.g., feedback signal) associated with the display, a distance of an EM radiation source and/or an image detector from an object, a target spectral-composition modification, and/or an intensity level of the EM radiation source. For example, a gain value can be calculated and used to modify an image-detector signal based on intensity level and a distance value associated with a distance between an EM radiation source and an object. In some embodiments, a gain value can be calculated based on an intensity value and further modified based on a feedback signal or signal associated with an environmental condition.

In some embodiments, the blocks in the flowchart can be executed in a different order. For example, a gain value can be calculated directly after an intensity-defining signal is received rather than after an image-detector signal is received.

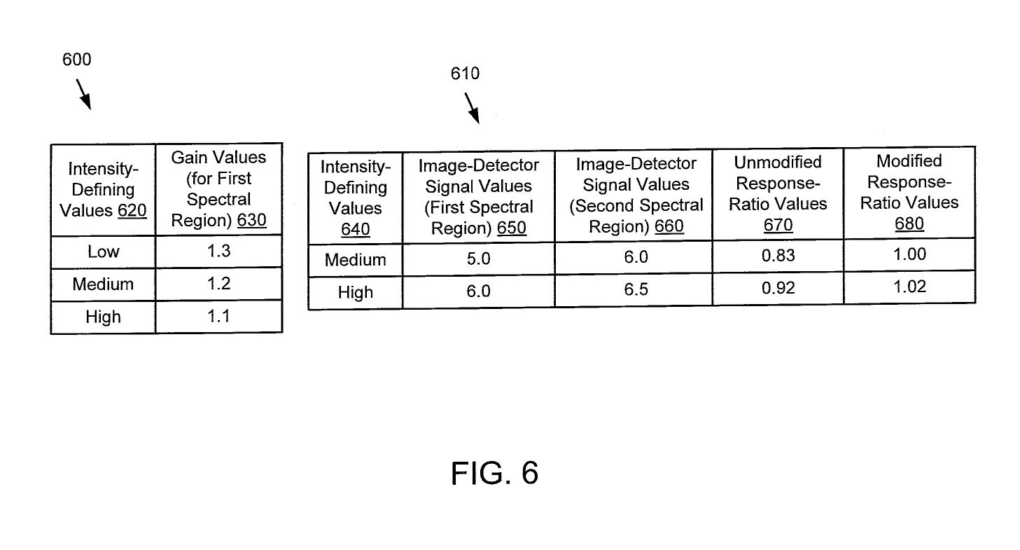

FIG. 6 is a schematic diagram that illustrates an example of a gain-intensity table 600 that can be used to modify an image-detector signal value associated with a first spectral region of EM radiation, according to an embodiment of the invention. The gain-intensity table 600 illustrates gain values (column 630) that are each associated with an intensity-defining value (column 620). The gain values (column 630) have been calculated such that a response-ratio value calculated based on a ratio of an image detector value associated with a first spectral region and an image detector value associated with a second spectral region will be at or near 1.0 over intensity values “Low” to “High” (shown in column 620).

Table 610 illustrates an example of image-detector signal values associated with the first spectral region of EM radiation (column 650) and image detector signal values associated with the second spectral region of EM radiation (column 660) at the intensity-defining values shown in column 640. Table 610 also illustrates that the unmodified response-ratio values (column 670) are below the target response-ratio value of 1.0. The unmodified response-ratio values are calculated based on the image-detector signal values associated with the first spectral region (column 650) before being modified using the gain values (column 630). After the image-detector signal values associated with the first spectral region (column 650) have been modified based on the gain values (column 630), the modified response-ratio values (column 670) are at or near the target response-ratio value of 1.0.

In an embodiment, a method includes changing an intensity level of electromagnetic radiation emitted from an electromagnetic radiation source towards an object based on an intensity-defining signal. A gain value is modified in response to the changing based on the intensity-defining signal. An image detector a signal associated with a spectral region of the electromagnetic radiation reflected from the object based on the gain value after the modifying is received.

In some embodiments, the gain value can be modified based on a gain-intensity relationship defined based on a response-ratio condition and based on the intensity-defining signal. In some embodiments, the gain value can be modified such that a response-ratio value calculated based on the signal and the intensity level satisfies a response-ratio condition associated with the spectral region and the image detector. In some embodiments, the electromagnetic radiation source and the image detector can be associated with a medical device. In some embodiments, the gain value can be modified based on an interpolation algorithm. In some embodiments, the intensity level can be changed by modifying a current to the electromagnetic radiation source, the intensity-defining signal is associated with the current. In some embodiments, the first gain value can be modified based on a first gain-intensity relationship associated with the first spectral region and the second gain value can be modified based on a second gain-intensity relationship associated with the second spectral region.

In some embodiments, the modification may occur in a piecewise linear manner. Thus, there can be a first set of gains for one intensity band and additional sets of gains for respective additional intensity bands. For example, the gain value can be a first gain value, the spectral region can be a first spectral region, and the signal can be a first signal. The method can also include a second gain value modified in response to the changing of the indicator of the intensity level. A second signal associated with a second spectral region of the electromagnetic radiation reflected from the object based on the second gain value can be received from the image detector. The second spectral region and the first spectral region can be different.

In another embodiment, a method can include receiving a first signal having an intensity value from an electromagnetic radiation source. A second signal can be produced based on electromagnetic radiation associated with a spectral region based on the intensity value such that a response-ratio value calculated based on the second signal satisfies a response-ratio condition associated with the spectral region.

In some embodiments, the second signal can be modified based on a gain value calculated based on a gain-intensity relationship defined based on the response-ratio condition. The method can also include detecting a change in at least one of the first signal or the intensity value. The gain value can be modified in response to the change based on the gain-intensity relationship.

In some embodiments, the electromagnetic radiation source can be associated with a dark environment. Alternatively, the electromagnetic radiation source can be associated with a variable light environment. In some embodiments, the electromagnetic radiation source and the image detector can be associated with a medical device or other consumer products. In some embodiments, the response-ratio condition can be based on a white balance response-ratio target value.

In yet another embodiment, an apparatus can include an electromagnetic radiation source configured to emit electromagnetic radiation having an intensity level towards an object. An image detector can be configured to produce a signal associated with a spectral region of the electromagnetic radiation reflected from the object. A processor can be in communication with the electromagnetic radiation source and the image detector. The processor can be configured to modify the signal based on a gain value associated with the spectral region to produce a modified signal such that a response-ratio value calculated based on the modified signal satisfies a response-ratio condition.

In some embodiments, the processor can be configured to trigger the intensity level of the electromagnetic radiation from the electromagnetic radiation source. In some embodiments, the processor can be configured to modify the gain value in response to a change in the intensity level of the electromagnetic radiation.

In some embodiments, the apparatus can also include a memory in communication with the processor and configured to store a gain-intensity relationship. The processor can be configured to calculate the gain value based on the gain-intensity relationship and based on the intensity level.

In another embodiment, a method can include detecting an image at a plurality of spectral regions based on electromagnetic radiation at each intensity level from a plurality of intensity levels, a first electromagnetic radiation having a spectral profile. A plurality of gain values can be stored where each gain value from the plurality of gain values can be associated with each spectral region from the plurality of spectral regions and associated with each intensity level from the plurality of intensity levels. The plurality of gain values can be associated with adaptive white balancing an imager when detecting a second electromagnetic radiation having a spectral profile substantially corresponding to the spectral profile of the first electromagnetic radiation.

Some embodiments relate to a computer storage product with a computer-readable medium (also referred to as a processor-readable medium) having instructions or computer code thereon for performing various computer-implemented operations. The media and computer code (also referred to as code) may be those specially designed and constructed for the specific purpose or purposes. Examples of computer-readable media include, but are not limited to: magnetic storage media such as hard disks, floppy disks, and magnetic tape; optical storage media such as Compact Disc/Digital Video Discs (“CD/DVDs”), Compact Disc-Read Only Memories (“CD-ROMs”), and holographic devices; magneto-optical storage media such as floptical disks; carrier wave signals; and hardware devices that are specially configured to store and execute program code, such as, but not limited to Application-Specific Integrated Circuits (“ASICs”), Field Programmable Gate Arrays (“FPGA's”), Digital Signal Processors (“DSPs”), Programmable Logic Devices (“PLDs”), and ROM and RAM devices. Examples of computer code include, but are not limited to, micro-code or micro-instructions, machine instructions, such as produced by a compiler, and files containing higher-level instructions that are executed by a computer using an interpreter. For example, an embodiment of the invention may be implemented using Java, C++, or other object-oriented programming language and development tools. Alternatively, an embodiments of the invention may be implemented using non-object oriented languages such as C or using Engineering or Mathematical Applications such as Matlab, MathCAD, Maple, etc. Additional examples of computer code include, but are not limited to, control signals, encrypted code, and compressed code.

In conclusion, certain embodiments of the invention provide, among other things, methods for spectral-composition control based on an intensity-defining signal associated with an electromagnetic radiation source. Those skilled in the art can readily recognize that numerous variations and substitutions may be made to achieve substantially the same results as achieved by the embodiments described herein. Accordingly, there is no intention to be limited to the disclosed exemplary forms. Many variations, modifications and alternative constructions are possible. Furthermore, aspects of different embodiments of the invention may be combined. It is intended that the specification and examples be considered as exemplary only, with a true scope and spirit of the invention being indicated by the following claims. cm What is claimed is:

1. A method for adjusting color channel signals based on spectral variations in the signal associated with a known source, comprising:
receiving a first signal having an intensity value from an electromagnetic radiation source; and
producing one or more second signals such that a response-ratio value calculated based on the one or more second signals satisfies a response-ratio condition associated with a spectral region of the first signal.

2. The method according to claim 1, further comprising producing an image based on the one or more second signals.

3. The method according to claim 2, wherein the spectral-composition of the image may vary based on a relative strength of the one or more second signals.

4. The method of claim 1, wherein an image detector is configured to receive the first signal and produce the one or more second signals.

5. The method of claim 4, wherein the image detector is associated with an endoscope.

6. The method of claim 1, further comprising modifying the one or more second signals in response to intensity level changes of the first signal.

7. The method of claim 1, further comprising modifying the one or more second signals in response to a gain-intensity relationship.

8. The method of claim 7, wherein the gain-intensity relationship is a function of the response-ratio condition.

9. The method of claim 8, wherein the response-ratio condition includes a target response ratio value and a range of response-ratio values.

10. A method for creating a gain-intensity relationship based on a response-intensity relationship, comprising:
emitting electromagnetic radiation toward an object at a first intensity level and at a second intensity level;
receiving a spectral region of the electromagnetic radiation reflected from the object at the first intensity and at the second intensity;
determining a response-ratio value at the first intensity level and a response-ratio value at the second intensity level to define a response-intensity relationship;
receiving a response-ratio condition; and
defining a gain-intensity relationship based on the response-intensity relationship and the response-ratio condition.

11. The method of claim 10, wherein the response-ratio condition is a target response-ratio condition.

12. The method of claim 10, wherein the response-ratio condition is a target range of response-ratio values.

13. The method of claim 10, wherein an electromagnetic source is configured to emit the electromagnetic radiation.

14. The method of claim 10, wherein an image detector is configured to receive the spectral region of the electromagnetic radiation.

15. A method for using a gain-intensity relationship, comprising:
changing an intensity level of an electromagnetic radiation source emitting electromagnetic radiation onto an object;
receiving an intensity defining signal associated with the intensity level of the electromagnetic radiation source;
receiving an image detector signal associated with a spectral region of electromagnetic radiation being reflected from the object; and
modifying the image detector signal based on a gain value associated with the image detector signal, the gain value being based on the indicator and the gain-intensity relationship.

16. The method of claim 15, further comprising calculating the gain value using the intensity defining signal prior to modifying the image detector signal.

17. The method of claim 16, wherein the calculating the gain value includes using a distance value associated with a distance between the electromagnetic radiation source and the object.

18. The method of claim 15, further comprising receiving the gain value prior to modifying the image detector signal.

19. The method of claim 15, further comprising modifying the gain value prior to modifying the image detector signal.

20. The method of claim 15, wherein the changing of the intensity level is triggered by a change in the intensity defining signal.
